(19) United States
(12) Patent Application Publication
(10) Pub. No.: US 2004/0210283 A1
(43) Pub. Date: Oct. 21, 2004

(54) Pump controller for body temperature regulatory system

(76) Inventors: Joseph Lorney Rose, Pembroke (CA); Lucien Potvin, Kanata (CA); Kevin Semeniuk, Ottawa (CA)

Correspondence Address: Clifford W. Browning, Bank one Center/Tower, 111 Monument Circle, Suite 3700, Indianapolis, IN, 46204-5137, US

(21) Appl. No.: 10/818,524

(22) Filed: Apr. 5, 2004

Related U.S. Application Data
(60) Provisional application No. 60/459,960, filed on Apr. 4, 2003.

(51) Int. Cl. F25D 17/02; F24F 11/04; A61F 7/12; A61F 7/00; F25B 21/02; G05D 23/00; F25D 23/12; F24F 11/06

(52) U.S. Cl. 607/96; 607/99; 62/185; 62/259.3; 165/247; 165/287

(57) ABSTRACT

An apparatus for supplying liquid to a heat exchanger for body temperature control for a human or animal comprises an inlet port for receiving liquid from a heat exchanger for body temperature control and for passing the liquid to a temperature conditioner to condition the temperature of the liquid. A pump is provided for pumping liquid from the temperature conditioner to the heat exchanger. The apparatus includes a sensor for sensing a parameter indicative of a condition of the liquid, for example, flow rate, pressure or temperature, and a controller for controlling the pump in response to the parameter.

### RELATED APPLICATIONS

This application claims priority from U.S. Provisional Application Serial No. 60/459,960, filed on 4^{th} Apr., 2003.

### FIELD OF THE INVENTION

The present invention relates to pump controllers, and in particular to pump controllers for controlling the rate of pumped fluid through a heat transfer garment for controlling the heat supplied to or drawn from a body.

### BACKGROUND OF THE INVENTION

Personal climate control systems are typically used by personnel exposed to extreme temperature environments to maintain their body temperature within a safe range to reduce the risk of injury, and to extend the period of time they can operate in such environments.

A known personal climate control system comprises a heat transfer vest which is worn as an undergarment and includes an array of tubes which carry liquid for restoring or removing heat to or from the body. A pump is connected to the vest for pumping liquid though the tubes to provide heating or cooling. Liquid from the vest is temperature conditioned by a suitable heat source or heat sink, for example, a bath of ice, before being recirculated. The pump and heat source/sink may be incorporated into a personal, portable unit which can be carried by an individual, so that cooling or heating may be provide while a person is working. The personal unit has a pump control switch which controls the pump to operate at one of two different pump speeds to control cooling or heating. In another system, the pump, heat source/sink and liquid supply are incorporated into a multi-person unit, having a number of liquid outlet and return ports, so that several people can be connected to the liquid supply at the same time. This multi-person system is used to provide body temperature regulation intermittently, by alternately connecting the unit to a person's heat transfer vest during a rest period and disconnecting the unit before resuming a work activity.

A drawback of these known systems is that the units are generally designed to operate with a particular heat transfer garment having certain impedance and heat transfer characteristics, so that if a different garment is to be used for a specific application, the whole system needs to be changed which can significantly increase the inventory of equipment required. Furthermore, personnel may engage in activities where the available source of electrical power is unknown or continuously changes, for example when changing between land-based and sea- or air-based operations. Moreover, it might not be possible to predict for how long a period temperature conditioning will be required or whether a battery source will have sufficient capacity. It might also not be possible to control or predict the cooling or heating power or capacity of the heat sink or source, which may continuously vary over time.

### SUMMARY OF THE INVENTION

According to one aspect of the present invention, there is provided an apparatus for supplying liquid to a heat exchanger for body temperature control, the apparatus comprising an inlet port for receiving liquid from an outlet port of a heat exchanger for body temperature control, and for passing the liquid to temperature conditioning means for conditioning the temperature of the liquid, a pump for pumping liquid from the temperature conditioning means and having an inlet port for receiving the liquid, an outlet port for supplying liquid from the pump to an inlet port of a heat exchanger for body temperature control, a sensor for sensing a parameter indicative of a condition of the liquid, and control means for controlling the pump in response to the parameter.

Advantageously, this liquid supply apparatus, which measures a condition of liquid in the system and controls the supply pump in response to the condition, significantly increases the sensitivity of the apparatus to real time changes during operation of the body temperature regulatory system for improved control.

In one embodiment, the sensor may be arranged to sense flow rate of the liquid through the system. As flow rate is dependent on the impedance or load connected to the system, advantageously, this arrangement allows the apparatus to be used for, and automatically adapt to different heat exchangers (e.g. heat exchange garments), without special knowledge or foreknowledge of the impedance characteristics of the particular heat exchange attached to the supply apparatus. As this arrangement is sensitive to load or impedance changes, it also allows the apparatus to detect a fault condition such as a blockage or other high impedance condition in the liquid circulatory system, or a leakage or other low impedance condition in the system.

In embodiments of the present invention, the flow rate sensor may comprise a sensor for sensing the rate of rotation of an electric motor driving the pump. Advantageously, this arrangement removes the need for special flow rate sensors positioned in the fluid circuit which are expensive and may add to the load on, and power drawn by the pump motor.

In embodiments of the present invention, the pump motor may comprise a DC electric motor and the sensor may be adapted to sense a signal generated by the motor during rotation thereof. For example, the signal may be that generated in one or more rotor or armature windings as the rotor rotates and alternately makes and breaks contact with the motor brushes. Advantageously, this arrangement derives a measure of the rotation rate based on a signal which is inherently generated by an electric motor and therefore precludes the need for more expensive sensors such as shaft encoders.

In embodiments of the present invention, the controller may be adapted to control the pump in response to the temperature of the liquid measured at one or more points in the circuit. For example either one or both of the outlet and inlet temperatures of the liquid may be measured, and/or the difference in temperature between the inlet, and outlet may be measured and the pump speed controlled in response thereto. Advantageously, this arrangement allows the flow rate through the heat exchanger to be adjusted dynamically depending on the amount of heating or cooling required (e.g. the heat load), and/or the cooling or heating capacity of the heat sink/heat source. This arrangement also allows the controller to automatically take into account changes in the available capacity of the heat source/heat sink and changes in the heat load.

In embodiments of the present invention, the pump may be powered from a source of stored electrical energy such as a battery. The apparatus may include a regulator or conditioner for regulating the power supplied from the battery to the pump. Advantageously, the conditioner may be adapted to limit the amount of power supplied to the pump to extend the time over which the pump can be driven and liquid supplied to the heat exchanger. In one embodiment, the conditioner may include switching means for intermittently switching power from the power source to generate a source of reduced power for driving the pump, and in one embodiment, the conditioner may include a pulse width modulator for converting DC power (or voltage) from the source of electrical energy to a reduced DC power (or voltage) supply signal for driving the pump. In one embodiment, the power may be limited to that which is available just prior to the abrupt drop in available power when the charge in a battery has been consumed.

According to another aspect of the present invention, there is provided an apparatus for supplying liquid to a heat exchanger for body temperature control, the apparatus comprising an inlet port for receiving liquid from an outlet port of a heat exchanger for body temperature control, and for passing the liquid to temperature conditioning means for conditioning the temperature of the liquid, a pump for pumping liquid from the temperature conditioning means and having an inlet port for receiving the liquid, an outlet port for supplying liquid from the pump to an inlet port of a heat exchanger for body temperature control, and control means having an input for receiving a signal indicative of a condition of a body in thermal contact with the heat exchanger and adapted for controlling the pump in response to the signal.

Advantageously, this arrangement provides a liquid supply apparatus for a body temperature regulatory system which is controlled in response to a measured condition of a body whose temperature is to be regulated, so that the heating or cooling rate can be adjusted dynamically depending on the measured condition(s). In embodiments of the present invention, the condition may be any one or more of skin temperature, core temperature, skin moisture, heart rate and respiration rate, as well as other conditions. The apparatus may include one or more values of any one or more conditions for comparing to a measured value and may be adapted to increase or decrease the pump speed (and flow rate) as a result of the comparison. The apparatus may further include means for storing a measured value or a series of values measured over time so that performance of the apparatus may be monitored. This may also allow the apparatus to “learn” or adapt itself to operate according to a particular cooling or heating requirement or to a number of different heating/cooling requirements.

According to another aspect of the present invention, there is provided an apparatus for supplying liquid to a heat exchanger for body temperature control, the apparatus comprising an inlet port for receiving liquid from an outlet port of a heat exchanger for body temperature control, and for passing the liquid to temperature conditioning means for conditioning the temperature of the liquid, a pump for pumping liquid from the temperature conditioning means and having an inlet port for receiving the liquid, an outlet port for supplying liquid from the pump to an inlet port of a heat exchanger for body temperature control, control means for generating a signal indicative of a predetermined value of electrical power for driving the pump, and regulator means responsive to the signal and having an input for receiving electrical power from a power source, and adapted to output electrical power to the pump which substantially corresponds to the predetermined value independently of changes in the electrical power from the power source.

According to another aspect of the present invention, there is provided an apparatus for supplying liquid to a heat exchanger for body temperature control, the apparatus comprising an inlet port for receiving liquid from an outlet port of a heat exchanger for body temperature control, and for passing the liquid to temperature conditioning means for conditioning the temperature of the liquid, a pump for pumping liquid from the temperature conditioning means and having an inlet port for receiving the liquid, an outlet port for supplying liquid from the pump to an inlet port of a heat exchanger for body temperature control, control means for generating a signal indicative of a predetermined value of electrical power for driving the pump, wherein the control means is adapted to limit the predetermined value to a value below a maximum power of a predetermined source of stored electrical energy.

According to another aspect of the present invention, there is provided a device for measuring the rate of rotation of an electric motor comprising detection means for detecting switching of a supply of electrical current to a coil of the motor as the rotor of the electric motor rotates and means for measuring the rate of rotation of the rotor based on the frequency of the switching.

According to another aspect of the present invention, there is provided an electric motor having a sensor for sensing switching of a supply of electrical current to a coil of the motor as the rotor of the electric motor rotates and which is arranged to output a signal indicative of the frequency of the switching.

According to another aspect of the present invention, there is provided a controller for controlling an electric motor comprising sensor means for sensing switching of a supply of electrical current to a coil of the motor as the rotor of the electric motor rotates, measuring means for measuring the frequency of the switching and control means for controlling the speed of the rotor in response to the measured frequency.

According to another aspect of the invention, there is provided a pump for pumping for pumping fluid through a heat exchanger for controlling heat supplied to or drawn from a body, the pump comprising a fluid input for receiving fluid from a fluid source, and a fluid output for supplying fluid to a heat exchanger, an electrical power input for receiving electrical power from a power source for driving the pump, and a controller adapted for supplying a predetermined value of electrical power to the pump which is independent of changes in the electrical power at the input.

According to another aspect of the present invention, there is provided a pump for pumping fluid through a heat transfer garment for controlling heat supplied to or drawn from a body, the pump comprising a fluid input for receiving fluid from a fluid source and a fluid output for supplying fluid to a garment, and a controller for controlling operation of the pump in response to a condition of the body.

According to another aspect of the present invention, there is provided a pump for pumping fluid through a heat transfer garment for controlling heat supplied to or drawn from a body, the pump comprising a fluid input for receiving fluid from a fluid source, and a fluid output for supplying fluid to a garment, and a controller for controlling the operation of the pump based on flow rate of the fluid through the garment.

According to another aspect of the present invention, there is provided a pump for pumping fluid through a garment for controlling heat supplied to or drawn from a body, the pump comprising a fluid input for receiving fluid from a fluid source and a fluid output for supplying fluid to a garment and a controller for controlling operation of the pump based on pressure of the fluid.

According to another aspect of the present invention, there is provided a pump for pumping fluid through a garment for controlling heat supplied to or drawn from a body, the pump comprising a fluid input for receiving fluid from a fluid source and an output for supplying fluid to the garment and a controller for controlling operation of the pump based on the temperature of fluid returned from the garment.

According to another aspect of the present invention, there is provided a pump for pumping fluid through a garment for controlling heat supplied to or drawn from a body, the pump comprising a fluid input for receiving fluid from a fluid source and an output for supplying fluid to a garment and a controller for controlling operation of the pump based on a temperature of the fluid at the output of the pump.

According to another aspect of the present invention, there is provided a method of controlling the temperature of the body of a human or animal, comprising placing a heat exchanger in thermal contact with the body, pumping liquid through the heat exchanger by means of a pump, measuring a condition of at least one of the liquid and the body, and controlling the pump in response to the measured condition.

According to another aspect of the present invention, there is provided a method of detecting rotation of an electric motor comprising detecting a signal generated by one or more armature windings of the motor as the motor rotates.

### BRIEF DESCRIPTION OF THE DRAWINGS

Examples of embodiments of the present invention will now be described with reference to the drawings, in which:—

FIG. 1 shows a block diagram of a pump controller according to an embodiment of the present invention;

FIG. 2 shows an embodiment of a sensor for sensing the rate of rotation of an electric motor, according to an embodiment of the present invention;

FIG. 3 shows an embodiment of a voltage regulator for use in embodiments of the present invention;

FIG. 4 shows a graph of battery terminal voltage (or power) as a function of time;

FIG. 5 shows a schematic diagram of an apparatus for supplying liquid to a heat exchanger for body temperature control according to an embodiment of the present invention; and

FIG. 6 shows a schematic diagram of a liquid supply apparatus according to another embodiment of the present invention.

### DESCRIPTION OF EMBODIMENTS

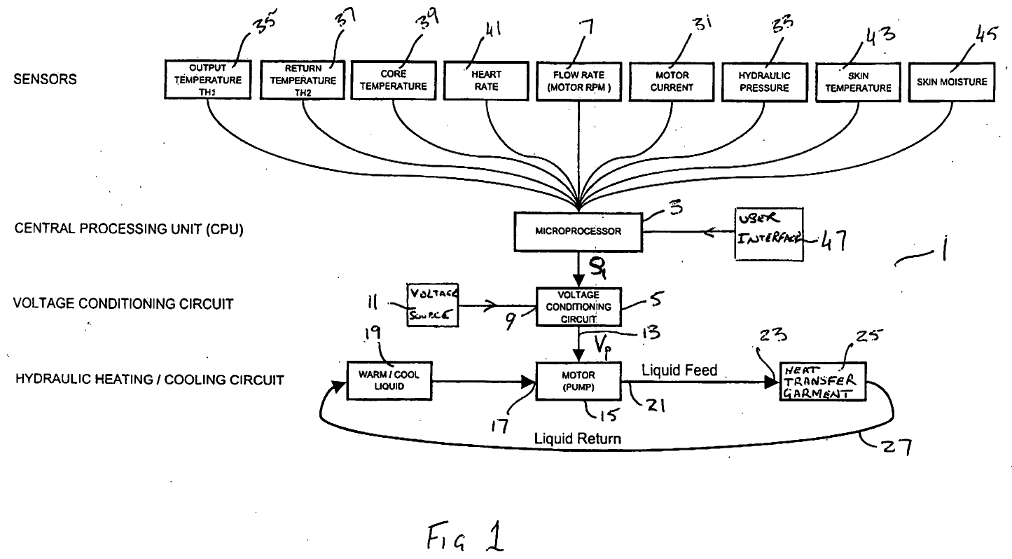

Referring to FIG. 1, a pump controller according to an embodiment of the present invention comprises a micro-processor 3, a voltage conditioning circuit 5 and a plurality of sensors, including a flow rate sensor 7. The voltage conditioning circuit 5 has an electrical power input 9 for receiving electrical power from a power source 11, and an electrical power output 13 for supplying controlled electrical power to the motor of an electrically driven pump 15. The pump 15 has a fluid inlet port 17 for receiving fluid from a fluid source 19, and a fluid outlet port 21 for supplying fluid to the inlet port 23 of a garment 25 for controlling the heat supplied to or drawn from the body of a user. A fluid return line 27 is connected to the fluid outlet port 27 of the garment 25 for returning fluid from the garment to the fluid source 19. The fluid source may comprise heat exchange means for cooling or heating the return fluid before being recirculated through the garment.

The flow rate sensor 7 is arranged to sense a parameter indicative of the flow rate of fluid in the fluid circuit between the fluid input and output of the pump 15 and to provide an indication of the flow rate to the processor 3. The flow rate of fluid through the circuit is one of the parameters that controls the rate at which heat is either transferred to or drawn from the body of a user. Advantageously, knowledge of actual flow rate through the circuit allows the heat transfer rate between the fluid and body to be determined, monitored and controlled more accurately and with greater certainty. Furthermore, a direct measurement of flow rate or of a parameter directly related thereto removes the need to rely upon indirect measurements such as may be deduced from the power or voltage supplied to the pump motor but which is not an exclusive variable on which flow rate depends. For example, the flow rate also depends on the impedance or load connected across the pump which may be unknown, or which may vary, for example, depending on the position of the user, which might result in changes in the cross-section of the circuit, or blockages in the circuit caused by intrusive material. Advantageously, knowledge of the actual flow rate also allows the controller to be readily adapted for use with different garments or different circuits having different impedances, and removes the need for knowledge of the impedance of any particular garment for controlling the heat transfer rate.

In a preferred embodiment, the flow rate is measured by sensing the rate of rotation of the pump electric drive motor, which is necessarily proportional to the flow rate of fluid at the output of the pump. Although in other embodiments, the flow rate sensor may comprise a flow rate meter which interacts directly with the fluid, measuring motor rate can be relatively simple to implement and does not interfere with the fluid flow by presenting an additional restriction. The rate of rotation of the motor may be sensed by any suitable means, for example, by using a shaft encoder. However, in a particularly advantageous embodiment, motor rate is measured by detecting a signal associated with each coil as the contact pairs of each coil alternately make and break contact with the brushes, as the rotor of the electric motor rotates. In practice, the signal generated by the motor may be relatively complex depending on its design and configuration (e.g. whether the armature is series or shunt wound, and the geometry of the brushes and winding commutator contacts, etc.). However, it has been found that certain characteristics of the signal repeat for each complete rotation of the armature or rotor, and may also repeat for each winding. Any substantially repeated signal generated during rotation of the rotor may be used to measure its rate of rotation. In one embodiment, the signal that is detected is the back electromotive force (e.m.f), or the counter electromotive force (c.e.m.f.) induced in the coil as the coil rotates past its position of field alignment with that of the stator. This signal is generated while the coil contacts (or commutator terminals) may still be in contact with the brushes, and therefore appears across the brushes. In a series wound armature, the signal may also be present at the brushes after the coil contacts leave the brushes, and the brushes contact the terminals of the next winding.

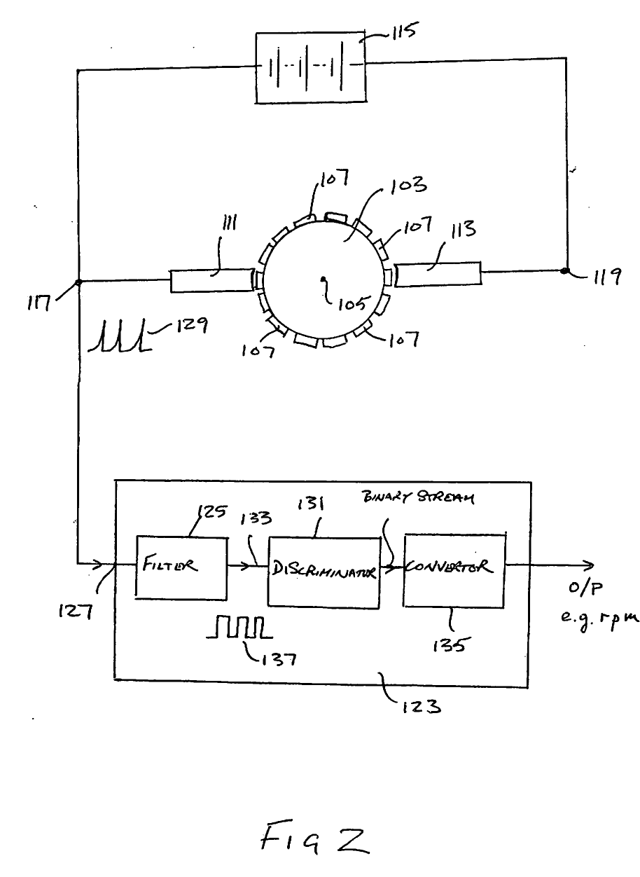

Advantageously, the signal, which may have the form of a pulse can be detected at an input terminal of the motor which supplies drive current to the or each motor coil via a brush. An example of a motor rate sensor according to an embodiment of the present invention which operates on this principle is described below with reference to FIG. 2.

FIG. 2 shows an arrangement for measuring the rate of rotation of the shaft of an electric motor, according to an embodiment of the present invention. FIG. 2 shows part of an electric motor, which includes a shaft 103 rotatably mounted about an axis 105 and having a plurality of pairs of commutator terminals 107 disposed about the shaft axis. The electric motor further comprises a pair of brushes 111, 113 which are suitably mounted to contact successive pairs of commutator terminals 107, as the shaft rotates. The electric motor is designed to be driven by a DC voltage source 115 which, in use, is connected across the input terminals 117, 119 connected to the brushes 111, 113.

A sensor 123 for measuring the rate of rotation of the motor shaft, according to an embodiment of the present invention, operates by detecting switching of DC current to each motor coil connected between pairs of commutator terminals 107 as the shaft rotates. In one embodiment, the switching is detected by detecting the back e.m.f (or c.e.m.f.) generated in an armature coil when the coil rotates past its position of field alignment with that of the stator, producing a voltage across the input terminals 117, 119. The e.m.f decays across the input terminal as the coil contacts rotate beyond their contact limit with the brushes, and as the brushes contact the terminals of the next winding segment. This combination of induced e.m.f and switching from one contact pair to another results in a voltage pulse, which occurs for each coil of the armature (or rotor) twice in each complete revolution of the armature (since each coil will contact the brushes every half revolution of the rotor with a corresponding reversal of current therethrough). The leading edge of the pulse may generally correspond to the back or counter e.m.f., and the trailing edge of the pulse may correspond to the e.m.f. decay. However, only the leading edge (or other characteristic of the signal) may be used to measure rotation rate.

Thus, the number of pulses in a complete revolution corresponds to the number of commutator terminals and therefore the rate of rotation is the pulse frequency divided by the number of commutator terminals (which is also equivalent to twice the number of coils or twice the number of pole pairs.

The rotation rate sensor may be used for any dc electric motor, which are typically characterised by having one or more armature windings to which current is supplied through a commutator whose operation is driven by rotation of the rotor itself.

A specific embodiment of the rotation rate sensor 123 comprises a filter 125 having an input 127 for receiving voltage pulses 129 from an input terminal 117 of the motor, a discriminator 131 connected to the output 133 of the filter 125, and a converter 135 connected to the output of the discriminator. The filter is adapted to remove noise and undesirable frequencies from the raw signal 129 to facilitate pulse detection, and, in particular to better define the leading and trailing edges and the peak height of each pulse, thereby producing, for example, a square wave pulse stream 137 at the output thereof. This pulse stream is passed to the discriminator which generates a “0” when the input signal is below a predetermined threshold (for example, ⅓ pulse height) and generates a “1” when the input signal is above a predetermined threshold (for example, ⅔ pulse height). Accordingly, the discriminator generates a binary pulse stream of ones and zeros which is passed to the input of the converter 135. In one embodiment, the converter detects the frequency of the pulses and converts this frequency to a value of rotation rate of the motor shaft by simply dividing the frequency by a constant which is equal to the number of commutator terminals. Alternatively, the converter may simply output a frequency corresponding to the pulse frequency, from which the rotation rate can be determined, or in another embodiment, the converter converts the rate of rotation to a value expressed in units of revolutions per minute (RPM), or any other measure of rotation rate.

Advantageously, the sensed motor rotation rate can be used by a controller to control the rate of rotation of the motor, allowing better control of a system that may be driven by the motor and improving control over the power consumed by the motor. For example, the sensed motor rate may be used by a motor controller to control the voltage supplied to the motor to maintain the motor rate at a controlled value, where the voltage supplied is below the available voltage from the power source. This may be particularly beneficial where the power source is a battery to reduce the current drawn therefrom, thereby extending battery life. As the load on the electric motor increases, the current drawn by the motor may also increase and the motor rotation rate may decrease. The ability to measure motor rate may be used to switch off the power source to the motor to prevent an excessive power draw from the source if the motor rate falls below a threshold value. A rate of rotation sensor according to embodiments of the present invention may be used in any application which employs an electric motor, including the temperature regulation system disclosed herein or any other application.

Returning to FIG. 1, an embodiment of the pump controller may further comprise a motor current sensor 31 for sensing the current drawn by the pump motor, and providing an indication of the sensed current to the microprocessor 3. The current sensor may comprise any suitable device for providing a signal indicative of motor current, and may, for example, comprise a resistor from which current is derived by measuring the voltage across the resistor or a current sensor integrated circuit (IC), which, for example, outputs a pulse train. The signal indicative of the sensed current provides an indication of the load on the motor and may be used to determine and/or monitor a condition of the pump and/or heat transfer system. For example, the processor 3 may be adapted to check whether the sensed current falls within a predetermined range of values which indicate normal operation. If the processor determines that the sensed current falls outside the normal range or that a current excursion outside the normal range continues for a predetermined length of time, the processor may indicate this condition to a user, and/or control the pump in response thereto. The upper and lower limits of current that define the range of normal operating conditions may either be preset and fixed, or varied by an operator or user, or “learned” by the processor during use of the pump and/or heat transfer system.

Motor current may be used either with, or without a flow rate measurement to detect a fault condition. For example, a motor current below the normal operating range may be caused by a reduction in pressure difference across the pump resulting, for example, from a lack of fluid condition (e.g. the presence of gas bubbles in the heat exchange liquid) or the presence of a leak in the system. Reduced operating pressure will also increase the speed of the pump which may also be detected by comparing the rate of rotation of the pump drive motor with a predetermined value which defines the upper limit of the range of normal operating rates.

A motor current above the normal operating range may be caused by an increase in impedance of the load presented to the pump and may be caused, for example, by a blockage or an incorrect hydraulic connection. The processor may be adapted to detect this condition by comparing the sensed current with a predetermined value which defines the upper limit of the normal operating range. An increase in impedance may also result in a reduction in pump speed which may be detected by the motor rotation rate sensor.

Although either current or motor rotation rate may be used to sense a fault condition, the processor may use the combination of both independent measurements to determine a fault condition, in order to increase its confidence that the type of fault has been identified correctly, so that appropriate action can be taken either by the processor to correct the fault or to identify the particular fault to a user.

The pump controller may be responsive to fluid pressure between the inlet port and outlet port of the heat exchange garment and may include a pressure sensor 33 for providing a signal indicative of the sensed pressure to the microprocessor 3. Advantageously, the provision of a pressure sensor (e.g. transducer) provides additional information which, with a measurement of fluid flow rate allows the impedance of a heat exchange garment to be determined. Sensing pressure allows the controller to adapt its control routine to different heat exchange garments having different hydraulic impedances.

The pump controller may be adapted to control the pump in response to temperature of the fluid. The embodiment of FIG. 1 comprises first and second temperature sensors 35, 37, one of which is arranged to measure the temperature (TH1) of fluid at the inlet of the heat transfer garment and the other is arranged to measure the temperature (TH2) of fluid at the outlet of the heat exchange garment. The temperature sensors may comprise any suitable temperature measuring device, for example, a thermistor or thermocouple. The controller may be adapted to monitor the temperature difference of the fluid between the inlet and outlet of the heat exchange garment and thereby determine a measurement of the normal differential temperature required or produced under operating conditions. The measurement of temperature difference in combination with measured flow rate may also be used by the controller to calculate the total heat load (in Watts).

The pump controller may be adapted to control the pump in response to the core temperature of the body of the person wearing the heat exchange garment. Referring to FIG. 1, the control system includes a core temperature sensor 39 for measuring the core temperature of the user and transmitting a signal indicative of the sensed temperature to the controller 1. The core temperature sensor may comprise any suitable device for measuring body temperature, for example, an ear temperature sensor, and ingestible pill transmitter or another device. The core temperature is monitored by the microprocessor 3 and is sensitive to any increase or decrease in heat stress, for example, due to user work rate. The controller may control the flow rate (which is proportional to the cooling/heating rate) to the user to control core temperature and to maintain the core temperature at a safe level.

The pump control system may be adapted to control the flow rate in response to the heart rate of a user. Referring to FIG. 1, this embodiment includes a heart rate sensor 41 which provides a signal indicative of heart rate to the controller 1. The heart rate sensor may comprise any suitable device, for example, a strain gauge or other compatible sensor. Pulses detected by the strain gauge or other pulse monitor are transmitted to the controller which may be adapted to detect any increase or decrease in thermal stress of a user based on the sensed heart pulse rate. In one embodiment, the controller may detect an increase or decrease in user work rate using a combination of both heart rate and core temperature according to an algorithm used by the microprocessor 3. If either the heart rate or core temperature, or both exceed a predetermined level, or exceed a predetermined level for a predetermined period of time, the controller may be adapted to adjust (e.g. increase) the flow rate to control the core temperature to a safe level.

The controller may be adapted to control the pump in response to skin temperature, for example, measured at different locations on the body. The embodiment of FIG. 1 includes a skin temperature sensor 43 which provides a signal indicative of skin temperature to the controller 1. The skin temperature sensor may comprise any suitable device, for example, a thermistor or thermocouple. The sensed skin temperature may be used by the controller to provide an indication of the thermophysiological condition of the user to adjust the flow rate. Measurements of skin temperature may be used in conjunction with other measurements to increase the reliability of the predicted or diagnosed physiological condition of the user.

In one embodiment, the pump controller may be adapted to control the pump in response to skin moisture. The embodiment of FIG. 1 includes a skin moisture sensor 45 which provides a signal indicative of skin moisture to the controller 1. The skin moisture sensor may comprise any suitable device, for example, a conductivity sensor. A measurement of skin moisture may be used by the controller to provide an indication of the thermophysiological condition of the user and may be used in conjunction with one or more other measurement to increase the reliability of the predicted or diagnosed physiological condition.

A measurement of the temperature difference of the fluid between the inlet and outlet of the heat exchange garment, together with the flow rate, which, for example, can be derived from the rate of rotation of the motor, allows the total thermal load to be calculated. In particular, the heat load is equal to temperature difference×specific heat of the liquid×flow rate.

Information relating to the thermophysiological condition of the user may be used to calculate the heating or cooling power (e.g. in Watts) required by the user and may, for example, be derived from an algorithm using the bio-feedback information from the sensors. For example, increased heart rate indicates an increase in user work rate and metabolic heat output. An increase in core temperature indicates the inability of the body to remove the increased metabolic heat load.

A measurement of motor rate and/or motor current may be used to indicate a fault in the system. In one embodiment, the controller may be adapted to generate a signal for an alarm in order to alert a user that the system is not ready for normal operation. The user can then remedy the situation before entering a potentially hazardous area with equipment that is not operating or connected properly. The controller may also generate an alarm signal if a failure is detected during operation of the system so that a user may take appropriate action, for example leave a potentially hazardous area.

Non-limiting examples of various possible operating modes for the controller are described below.

System Operation

Manual Mode

In one embodiment, the control system may include a user interface, for example, the user interface 47 shown in FIG. 2, for receiving user inputs. For example, the user interface 47 may comprise a selector switch which allows a user to select a heating or cooling rate which sets the pump motor at a particular, constant operating speed. In one example, the microprocessor receives the selected cooling rate from the user interface 47 and outputs a signal S₁ to the voltage conditioning circuit of constant value. The voltage conditioning circuit 5 outputs a constant voltage Vₚ to the motor pump regardless of voltage variations in the source voltage at its input 9.

Semi-Automatic Mode

In semi-automatic mode, the controller may be adapted to permit a user to select a heating or cooling rate (e.g. in Watts). The controller is arranged to control the flow by controlling the signal S₁ at the output of the microprocessor based on a heat calculation algorithm (TH1−TH2×specific heat of the liquid×flow rate), in order to maintain the selected heating/cooling rate.

Bio-Feedback Operating Mode 1

In an example of a first operating mode using bio-feedback, flow is regulated to provide a specific heating/cooling value calculated by a bio-feedback algorithm in the microprocessor, based on average physiological variables. In one example, the algorithm uses data values from any one or more of the heart rate, core temperature, differential temperature and flow rate sensors to generate a control signal S₁ for controlling the power to the pump motor via the voltage conditioning circuit 5. Again, the voltage conditioning circuit 5 allows the pump motor to faithfully follow and adhere to the commanded flow rate control signal S₁ regardless of variations or changes in the source voltage at its input 9.

Operating Mode With Bio-Feedback 2

In another mode of operation, using bio-feedback, flow is regulated to provide a specific heating/cooling value calculated by a bio-feedback algorithm based on physiological variables determined for a specific user. In one embodiment, the algorithm uses data values from any one or more of the heart rate and core temperature sensors, the first and second temperature sensors and the flow rate sensor, and the microprocessor 3 outputs a control signal S₁ based on the measured value(s).

In other modes of operation, any one or more of the sensors described above, for example, and shown in FIG. 1 may be used to control the flow rate.

The program used to control the microprocessor may be stored in a suitable memory, for example, a ROM or a RAM and the program may be stored in such a way that it can be erased or otherwise removed and replaced by another program, as required. The controller may further comprise a memory for storing data from one or more sensors, either for use in controlling the flow rate or as a record of operation of the pump and heat transfer garment, for example, for subsequent analysis. In this case, the data may advantageously be time (and possibly date) stamped to provide a record of changes in operation of the system over time.

Although the controller of the embodiment of FIG. 1 may comprise a microprocessor, other embodiments may comprise other forms of processor, or other digital or analogue circuitry to generate the required pump control signal.

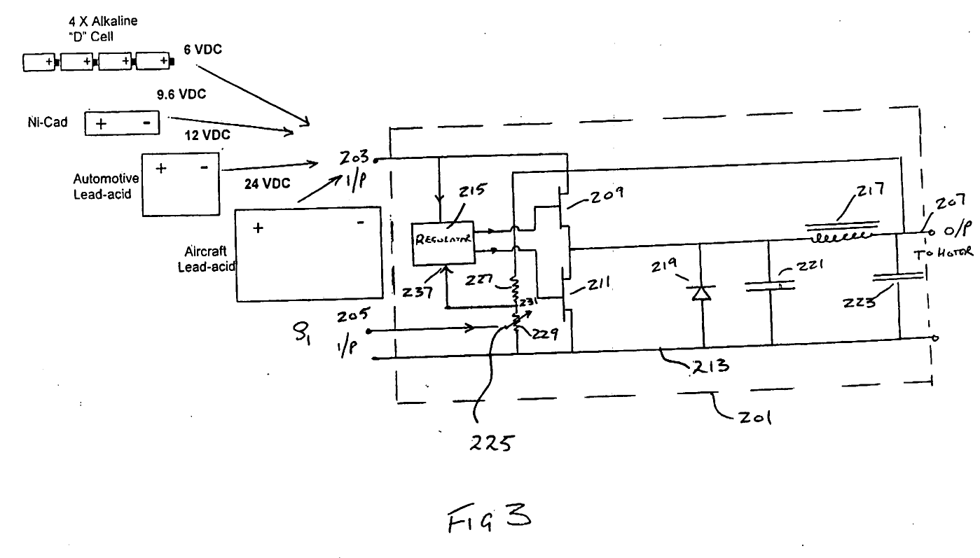

An example of a voltage conditioning circuit which may be used, for example, in the embodiment of FIG. 1 to maintain the power supply to the pump motor at the level determined by the microprocessor, independently of the source voltage is shown in FIG. 3. Referring to FIG. 3, the voltage conditioning circuit 201 has an input 203 for receiving a source voltage, a control input 205 for receiving a control signal, for example, from the microprocessor of FIG. 1, and an output 207 for outputting a required voltage to the pump motor. The voltage conditioning circuit 201 includes a synchronous buck switching regulator circuit which includes first and second series connected field effect transistors (FET) 209, 211, connected between the input 203 and a ground rail 213, and whose switching operation is controlled by a synchronous buck regulator 215. An inductor 217 is connected between the source and drain of the first and second FETs 209, 211, and the other side of the inductor is connected to the output 207. A diode and capacitor 219, 221 are connected in parallel between the source and drain of the first and second FETs and the ground rail 213, and a second capacitor 223 is connected between the output 207 and the ground rail 213. The circuit includes a potential divider 225 comprising two resistors 227, 229, one of which is a variable resistor, and whose resistance can be controlled by a control signal received at the control signal input 205. The junction 231 between the resistors 227, 229 is connected to an input 233 of the synchronous buck regulator 215. The potential divider circuit is connected between the output 207 and the common ground rail 213. In operation, the synchronous buck regulator controls the timing of the switching of the field effect transistors 209, 211 to charge and discharge the inductor 217 at a rate which converts the input voltage to the required output voltage, as determined by the control signal at the control input 205. If the input voltage varies, the synchronous buck regulator varies the FET switching rate to maintain the output voltage at the level set by the control signal S₁.

Advantageously, the voltage conditioning circuit allows the pump controller to be used with different voltage sources without any need for adjustment. For example, in one embodiment, the pump controller may be adapted for use with any DC voltage source ranging from 3 to 32 volts or more, or a voltage less than 3 volts.

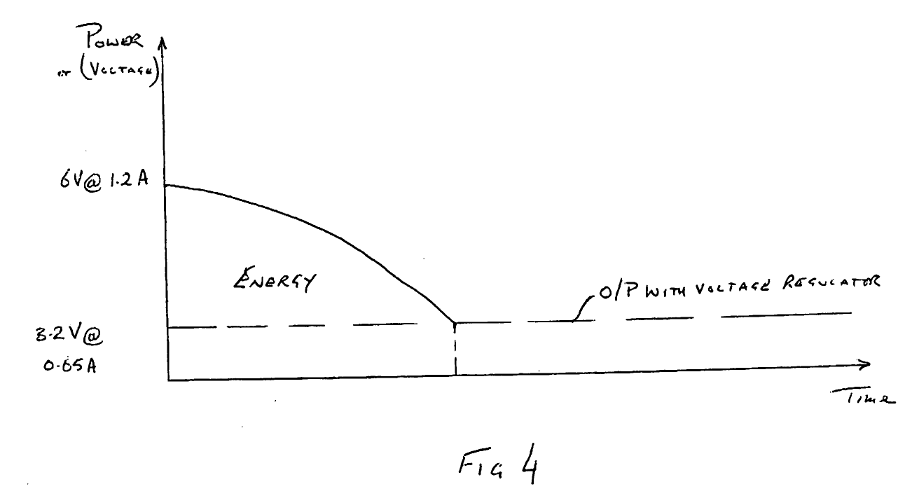

Generally, the voltage output of a battery decreases over time, as the battery is used. Advantageously, the voltage conditioning circuit allows the pump to operate at a controlled rate regardless of the change in battery voltage. In one embodiment, the controller may set a maximum level of voltage for driving the pump which is less than the maximum voltage output by a battery source at the beginning of its life. Advantageously, this reduces the maximum available current that can be drawn from the battery source thereby extending the time over which the stored electrical energy is consumed. In a specific embodiment, the voltage limit may be set at substantially the voltage output by the battery at the end of its life just before the current falls to zero. Advantageously, not only does this extend battery life, but also ensures that this level of voltage is always available from the battery source over its life, and thereby ensures that a predetermined flow rate, and thus heat or cooling rate, is continuously available. Therefore, this arrangement can extend battery life considerably so that the temperature regulation system can be used over extensive periods of time without replacing or recharging the power source. For example, in one embodiment, the power source comprises a plurality, for example, four D-cell batteries, each having an initial voltage output of 1.6 volts for a total voltage of 6.4 volts. At the end of battery life, the initial voltage of each battery drops to approximately 0.8 volts or to a total voltage of 3.2 volts. An example of a curve showing battery voltage depletion over time is illustrated in FIG. 4.

The final terminal voltage may be used to determine the number of batteries required for a particular application. For example, in one embodiment, the heat transfer system may be designed for a maximum voltage requirement of 3.2 volts, in which case four D-cell batteries will be sufficient. As illustrated in FIG. 4, the use of a voltage regulator that provides an output of 3.2 volts substantially extends battery life. An embodiment of a thermal regulatory system using such a power source has been found in tests to be capable of running for up to about 35 hours, which is considerably more than the expected running time of 5 hours without the regulator/conditioner.

Advantageously, the use of a voltage conditioning circuit which is separate from a control processor removes the need for additional processing by the processor in order to compensate for connection of the system to different source voltages or variations over time in the voltage from the same voltage source. The ability to accurately control the voltage supplied to the pump motor irrespective of changes in source voltage gives the system a repeatable heat transfer rate at consistent levels over a wide source voltage range. The pump controller also removes the requirement of providing different pump motors for different power sources so that the same heat transfer apparatus may be used with a variety of power sources, for example, portable power sources, or power supplies used in vehicles, such as land or water vehicles or aircraft including winged aircraft and helicopters.

In other embodiments of the present invention, the voltage conditioner may be controlled to output a predetermined voltage by any suitable means other than a processor, so that a processor is not required. The ability of the voltage conditioner to maintain the output voltage at the controlled, predetermined level over a range of input voltage values, also removes the need for a processor which could otherwise perform this task. For example, in one embodiment, the predetermined voltage may be controlled directly by a switch that is operable by a user, rather than by or via a processor.

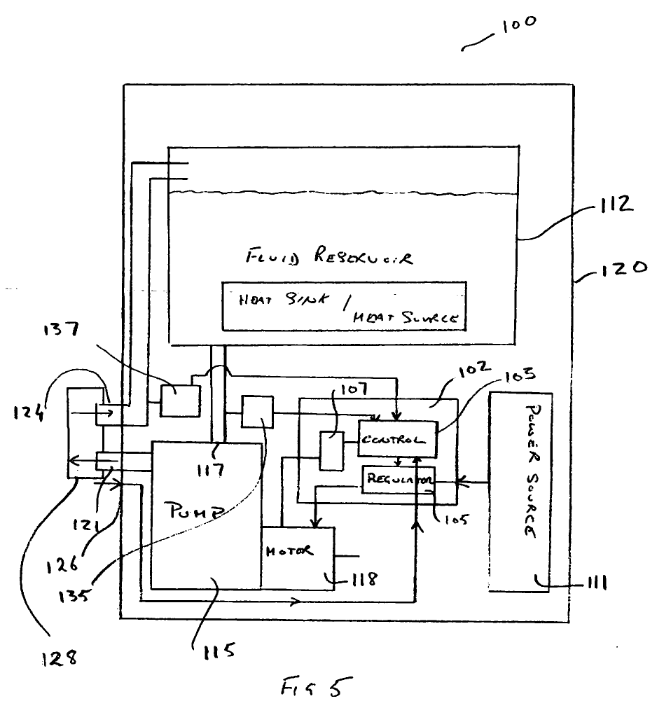

FIG. 5 shows an apparatus for supplying liquid to a heat exchanger for body temperature control according to another embodiment of the present invention. The apparatus, generally indicated at 100, comprises a control system 102 which includes a pump controller 103, a regulator 105 and a flow rate sensor 107. The apparatus further comprises a container 112 for containing fluid, e.g. liquid, and a pump 115 having an inlet port 117 connected to the container 112 and an outlet port 121. In this embodiment, the pump is driven by an electric motor 118 from a source of electrical power 111, such as one or more batteries. An inlet port 124 is provided to return liquid from a heat exchanger to the container 112, and temperature sensors 135, 137 are provided to measure the output temperature and return temperature of the fluid, respectively. One or more other sensors may also be provided, for example, to measure motor current or hydraulic/fluid pressure as described above in connection with FIG. 1. A sensor signal inlet/outlet port 126 may also be provided for receiving signals from one or more external sensors, for example, a sensor which measures a condition of the body whose temperature is to be regulated, and may include any one or more of the body condition sensors disclosed herein.

The fluid inlet and outlet ports are adapted for connection to a fluid line for carrying fluid to a heat exchanger, and may include any suitable connector 128, such as a quick-release connector. One or more of the aforementioned components may conveniently be enclosed within a housing 120, which may have a carrying handle and/or may be adapted to be worn by a person or individual. In use, a heat sink or heat source may be provided to cool or heat the fluid and may, for example, comprise a frozen liquid, such as ice, placed within the container. Alternatively, the heat source/sink may simply comprise a body of stored liquid, which may be the same liquid that is circulated through the heat exchanger (not shown). In other embodiments, the heat source/sink may comprise a heater or a cooler such as a refrigerator unit.

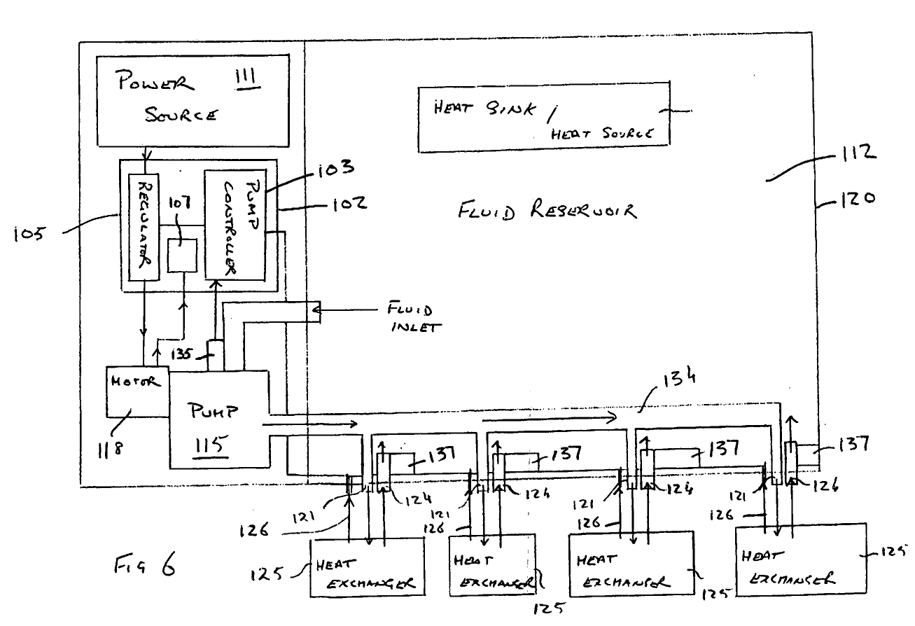

FIG. 6 shows another embodiment of an apparatus for body temperature control, which is similar to the embodiment shown in FIG. 5, and like parts are designated by the same reference numerals. The main difference between these embodiments is that the apparatus shown in FIG. 6 has a plurality of fluid inlet/outlet ports 121, 124, so that the apparatus can supply heat to a plurality of heat exchangers 125 at the same time. One or more temperature sensors 137 may also be provided to measure the temperature of return fluid to one or more inlet port 124. A plurality of sensor signal inlet/outlet ports 126 may also be provided for receiving signals from one or more sensors for measuring a condition associated with each body whose temperature is being controlled, and which are passed to the pump controller 102. In this embodiment, each fluid outlet port 121 is connected to a pump 115 via a manifold 134. As fluid is delivered to each outlet port 121 by the same pump 115, the load connected to the pump will depend on the number of heat exchangers connected thereto at any one time. As the outlet ports are connected to the pump in parallel, the load or impedance will generally decrease as the number of connected heat exchangers increases. In one embodiment, the connection or disconnection of a heat exchanger to the heat transfer fluid supply apparatus may be detected by the flow rate sensor 107, for example, as an increase or decrease, respectively, in flow rate, and this information may be used by the pump controller to control the power supplied to the pump motor.

Alternatively, or in addition to, the connection or disconnection of a heat exchanger to the apparatus may be detected by some other means, for example by a sensor associated with the connector which connects the inlet/outlet ports to the heat exchanger to detect whether an actual physical connection to the heat exchanger has been made. This information may be passed to the pump controller which may then control the pump motor (and flow rate) in response thereto. For each number of heat exchangers connected to the apparatus, the apparatus may include a value or range of values of acceptable flow rates for each number which may be used by the pump controller to control the pump speed to within acceptable levels. For example, the measured flow rate may be compared with one or more threshold values (for example, at the upper and lower limits of each range) and the pump controller may control the power to the motor to maintain the flow rate within the range.

The return temperature of the fluid for each heat exchanger may also be monitored and used by the pump controller to control the flow rate through the heat exchangers. For example, if the return fluid from one or more heat exchangers is above a certain threshold, or the difference between the fluid from the pump and the return fluid is above a predetermined threshold, the pump controller could use this information to increase the flow rate, if cooling is required. On the other hand, if the return temperature of the fluid or the difference between the outlet and return temperatures is below a predetermined threshold, the pump controller may use this information to reduce the flow rate.

The pump controller may control the flow rate in response to a condition of the body whose temperature is being regulated through use of a heat exchanger.

Any embodiments of the apparatus may be used with any form of suitable heat exchanger, including fluid heat transfer garments for humans or for animals, or simple heat transfer covers or blankets for use in controlling the body temperature of humans or animals.

Modifications and changes to the embodiments described above will be apparent to those skilled in the art and any feature described in relation to one embodiment may be incorporated with any other embodiment.

1. An apparatus for supplying liquid to a heat exchanger for body temperature control, the apparatus comprising:
an inlet port for receiving liquid from an outlet port of a heat exchanger for body temperature control, and for passing said liquid to temperature conditioning means for conditioning the temperature of said liquid;
a pump for pumping liquid from said temperature conditioning means and having an inlet port for receiving said liquid;
an outlet port for supplying liquid from the pump to an inlet port of a heat exchanger for body temperature control;
a sensor for sensing a parameter indicative of a condition of said liquid; and
control means for controlling the pump in response to said parameter.

2. An apparatus as claimed in claim 1, wherein said condition is the flow rate of said liquid.

3. An apparatus as claimed in claim 2, further comprising an electric motor for driving said pump, and wherein said parameter is based on the speed of said motor.

4. An apparatus as claimed in claim 3, wherein said electric motor comprises a rotary motor and said sensor is adapted to sense switching of a supply of electrical current to a coil of said motor as the rotor of said electric motor rotates.

5. An apparatus as claimed in claim 4, further comprising measuring means for measuring the frequency of said switching, and wherein said control means is arranged to control said pump in response to the measured frequency.

6. An apparatus as claimed in claim 4, wherein said sensor is adapted to detect electrical pulses produced by said switching.

7. An apparatus as claimed in claim 6, further including filter means for receiving said pulses and removing frequencies above a predetermined frequency and for transmitting the filtered pulses to said sensor.

8. An apparatus as claimed in claim 6, further comprising filter means for receiving said pulses and for removing frequencies below a predetermined frequency and for passing the filtered pulses to said sensor.

9. An apparatus as claimed in claim 1, wherein said condition comprises the flow rate of said liquid, and said control means is adapted to control the speed of the pump in response to said parameter.

10. An apparatus as claimed in claim 1, wherein said condition comprises pressure of said liquid, and said control means is adapted to control the speed of the pump in response to said parameter.

11. An apparatus as claimed in claim 1, wherein said condition comprises temperature of said liquid, and said control means is adapted to control the speed of the pump in response to said parameter.

12. An apparatus as claimed in claim 11, wherein said parameter is one of the temperature of the liquid at the outlet port of said apparatus, the temperature of the liquid at the inlet port which receives liquid from the heat exchanger, and the difference between the temperature of the liquid at the outlet port of said apparatus and the temperature of the liquid at the inlet port which receives liquid from the heat exchanger.

13. An apparatus as claimed in claim 1, wherein said condition is flow rate and said apparatus comprises an electric motor for driving said pump and switching means responsive to said parameter for switching off electrical power to said motor if said parameter exceeds a predetermined value.

14. An apparatus as claimed in claim 1, wherein said condition is flow rate and said apparatus comprises an electric motor for driving said pump and switching means responsive to said parameter for switching off electrical power to said motor if said parameter falls below a predetermined value.

15. An apparatus as claimed in claim 1, wherein said condition is pressure and said apparatus comprises an electric motor for driving said pump and switching means responsive to said parameter for switching off electrical power to said motor if said parameter exceeds a predetermined value.

16. An apparatus as claimed in claim 1, wherein said condition is pressure and said apparatus comprises an electric motor for driving said pump and switching means responsive to said parameter for switching off electrical power to said motor if said parameter falls below a predetermined value.

17. An apparatus as claimed in claim 1, further comprising storage means arranged for storing at least one value of said parameter.

18. An apparatus as claimed in claim 17, wherein said parameter comprises any one of flow rate, pressure of said liquid, the temperature of the liquid at said outlet port, the temperature of the liquid at the inlet port which receives liquid from said heat exchanger, and the difference between the temperature of the liquid at said outlet port receives liquid from said heat exchanger.

19. An apparatus as claimed in claim 1, further comprising an electric motor for driving said pump, and wherein said control means is adapted to control said pump in response to electrical current drawn by said motor.

20. An apparatus as claimed in claim 19, further comprising switching means arranged to switch off electrical power to said motor when said current exceeds a predetermined value.

21. An apparatus as claimed in claim 19, further comprising switching means arranged to switch off electrical power to said motor when said motor current falls below a predetermined value.

22. An apparatus as claimed in claim 19, further comprising storage means for storing at least one value of current drawn by said motor.

23. An apparatus as claimed in claim 1, wherein said control means includes input means for receiving a signal indicative of a condition of a body in thermal contact with said heat exchanger and is adapted to control said pump in response to said condition.

24. An apparatus as claimed in claim 23, wherein said condition comprises any one of more of a value of an internal temperature of said body, a temperature of the exterior of said body, heart rate and moisture on the exterior of said body.

25. An apparatus as claimed in claim 1, further comprising a container for containing said temperature conditioning means.

26. An apparatus as claimed in claim 25, wherein said temperature conditioning means comprises one of a heat sink and a heat source.

27. An apparatus as claimed in claim 26, wherein said heat sink comprises frozen liquid.

28. An apparatus as claimed in claim 27, wherein said heat source comprises at least one of a heater and thermal energy storage means.

29. An apparatus as claimed in claim 1, further comprising said temperature conditioning means.

30. An apparatus as claimed in claim 29, wherein said temperature conditioning means comprises electrically driven refrigeration means.

31. An apparatus as claimed in claim 1, further comprising connector means for connecting said inlet port to a heat exchanger.

32. An apparatus as claimed in claim 1, further comprising connector means for connecting said outlet port to a heat exchanger.

33. An apparatus as claimed in claim 1, further comprising regulator means for receiving electrical power for driving said pump and for controlling the electrical power supplied to said pump.

34. An apparatus as claimed in claim 33, wherein said regulator means is adapted for supplying a predetermined value of electrical power to said pump which is independent of changes in the electrical power received by said regulator means.

35. An apparatus as claimed in claim 34, wherein said control means is adapted to generate a signal indicative of said value of electrical power for driving said pump, and said regulator means includes an input for receiving said signal.

36. An apparatus as claimed in claim 35, further comprising a selector for enabling a user to select a value of electrical power for driving said pump from a plurality of values and wherein said signal is based on said selected value.

37. An apparatus as claimed in claim 33, further comprising limiting means for limiting the voltage at the output of said regulator means to or below a predetermined value.

38. An apparatus as claimed in claim 37, wherein said predetermined value is less than the maximum value of a source of stored electrical energy.

39. A pump as claimed in claim 38, wherein said predetermined value substantially corresponds to the voltage of a battery source at which said current falls to zero.

40. An apparatus as claimed in claim 33, wherein said control means comprises processor means for determining the value of electrical power to said pump based on any one or more of a measured flow rate of said fluid, a measured electrical current to said pump, a pressure of said fluid, a temperature of said fluid, a temperature of said body, an internal temperature of said body, an external temperature of said body, moisture on said body, heart rate and respiratory rate, and any other parameter indicative of a condition of said body.

41. An apparatus for supplying liquid to a heat exchanger for body temperature control, the apparatus comprising:
an inlet port for receiving liquid from an outlet port of a heat exchanger for body temperature control, and for passing said liquid to temperature conditioning means for conditioning the temperature of said liquid;
a pump for pumping liquid from said temperature conditioning means and having an inlet port for receiving said liquid;
an outlet port for supplying liquid from the pump to an inlet port of a heat exchanger for body temperature control, and
control means having an input for receiving a signal indicative of a condition of a body in thermal contact with said heat exchanger and adapted for controlling the pump in response to said signal.

42. An apparatus for supplying liquid to a heat exchanger for body temperature control, the apparatus comprising:
an inlet port for receiving liquid from an outlet port of a heat exchanger for body temperature control, and for passing said liquid to temperature conditioning means for conditioning the temperature of said liquid;
a pump for pumping liquid from said temperature conditioning means and having an inlet port for receiving said liquid;
an outlet port for supplying liquid from the pump to an inlet port of a heat exchanger for body temperature control;
control means for generating a signal indicative of a predetermined value of electrical power for driving the pump, and
regulator means responsive to said signal and having an input for receiving electrical power from a power source, and adapted to output electrical power to said pump which substantially corresponds to said predetermined value independently of changes in the electrical power from said power source.

43. An apparatus as claimed in claim 42, further comprising limiting means for limiting the voltage at the output of said regulator means to or below a predetermined value.

44. An apparatus as claimed in claim 43, wherein said predetermined value is less than the maximum value of a predetermined source of stored electrical energy.

45. An apparatus as claimed in claim 44, wherein said predetermined value substantially corresponds to the voltage of a battery source at which said current falls to zero.

46. An apparatus for supplying liquid to a heat exchanger for body temperature control, the apparatus comprising:
an inlet port for receiving liquid from an outlet port of a heat exchanger for body temperature control, and for passing said liquid to temperature conditioning means for conditioning the temperature of said liquid;
a pump for pumping liquid from said temperature conditioning means and having an inlet port for receiving said liquid;
an outlet port for supplying liquid from the pump to an inlet port of a heat exchanger for body temperature control;
control means for generating a signal indicative of a predetermined value of electrical power for driving the pump, wherein said control means is adapted to limit said predetermined value to a value below a maximum power of a predetermined source of stored electrical energy.

47. An apparatus as claimed in claim 46, further comprising regulator means having an input for receiving dc electrical power from a dc source, and switching means for switching said dc power to generate an output power of less than the power at said input.

48. An apparatus as claimed in claim 46, wherein said switching means is arranged to generate a pulse stream from said dc power input.

49. An apparatus as claimed in claim 48, further comprising means for controlling at least one of the pulse width and the pulse interval of said stream.

50. A device for measuring the rate of rotation of an electric motor comprising detection means for detecting switching of a supply of electrical current to a coil of said motor as the rotor of said electric motor rotates and means for measuring the rate of rotation of said rotor based on the frequency of said switching.

51. A device as claimed in claim 50, wherein said detection means is adapted to detect electrical pulses produced by said switching and said measuring means is arranged to measure the rate of rotation of said rotor based on the frequency of said pulses.

52. An electric motor having a sensor for sensing switching of a supply of electrical current to a coil of said motor as the rotor of said electric motor rotates and which is arranged to output a signal indicative of the frequency of said switching.

53. An electric motor as claimed in claim 52, wherein said sensor is adapted to sense electrical pulses produced by said switching.

54. An electric motor as claimed in claim 52, further comprising measuring means for measuring the frequency of said switching.

55. An electric motor as claimed in claim 52, further comprising a controller for controlling the electrical power supplied to drive said motor in response to the measured frequency.

56. A controller for controlling an electric motor comprising sensor means for sensing switching of a supply of electrical current to a coil of said motor as the rotor of said electric motor rotates, measuring means for measuring the frequency of said switching and control means for controlling the speed of said rotor in response to the measured frequency.

57. A controller as claimed in claim 56, wherein said sensor means is adapted to detect electrical pulses produced by rotation of said rotor.

58. A controller as claimed in claim 27 or 28, wherein each of said pulses is produced by the back electromotive force of said coil as said rotor rotates.

59. A pump for pumping for pumping fluid through a heat exchanger for controlling heat supplied to or drawn from a body, the pump comprising a fluid input for receiving fluid from a fluid source, and a fluid output for supplying fluid to a heat exchanger, an electrical power input for receiving electrical power from a power source for driving said pump, and
a controller adapted for supplying a predetermined value of electrical power to said pump which is independent of changes in the electrical power at said input.

60. A pump as claimed in claim 59, wherein said controller includes an input for receiving a signal indicative of said predetermined electrical power.

61. A pump as claimed in claim 60, further comprising a selector for enabling a user to select a value of electrical power for driving said pump from a plurality of values.

62. A pump as claimed in claim 59, further comprising limiting means for limiting the voltage at the output of said controller to a predetermined value.

63. A pump as claimed in claim 59, wherein said predetermined value is less than the maximum value of a source of stored electrical energy.

64. A pump as claimed in claim 63, wherein said predetermined value substantially corresponds to the voltage of a battery source at which said current falls to zero.

65. A pump as claimed in claim 59, further comprising processor means for determining the value of electrical power to said pump based on any one or more of a measured flow rate of said fluid, a measured electrical current to said pump, a pressure of said fluid, a temperature of said fluid, a temperature of said body, an internal temperature of said body, an external temperature of said body, moisture on said body, heart rate and respiratory rate, and any other parameter indicative of a condition of said body.

66. A pump for pumping fluid through a heat exchanger for controlling heat supplied to or drawn from a body, the pump comprising a fluid input for receiving fluid from a fluid source and a fluid output for supplying fluid to a heat exchanger, and a controller for controlling operation of said pump in response to a condition of said body.

67. An apparatus as claimed in claim 1, wherein said control means is adapted to control said pump in response to pressure difference between two points in a fluid circuit to which said pump is connected.

68. A method of controlling the temperature of the body of a human or animal, comprising placing a heat exchanger in thermal contact with said body, pumping liquid through said heat exchanger by means of a pump, measuring a condition of at least one of said liquid and said body, and controlling said pump in response to the measured condition.

69. A method as claimed in claim 68, wherein said condition comprises at least one of flow rate of said liquid, a temperature of said liquid, a pressure of said liquid, a temperature of said body, a core temperature of said body, a skin temperature of said body, an amount of moisture on said body, a heart rate and a respiration rate.

70. A method of detecting rotation of an electric motor comprising detecting a signal generated by one or more armature windings of the motor as the motor rotates.

71. A method as claimed in claim 70, comprising deriving a measure of the rate of rotation of said electric motor based on the frequency of repetition of said signal.
